(19) United States
(12) Patent Application Publication
(10) Pub. No.: US 2023/0157302 A1
(43) Pub. Date: May 25, 2023

(54) METHODS FOR INHIBITING FUSARIUM MYCOTOXIN PRODUCTION

(71) Applicant: Institut National de Recherche Pour L'Agriculture, L'Alimentation et L’Environnement, Paris (FR)

(72) Inventors: ALEJANDRO CABEZAS-CRUZ, ALFORTVILLE (FR); FLORENCE FORGET, LATRESNE (FR); VESSELA ATANASOVA, BORDEAUX (FR)

(21) Appl. No.: 17/920,060

(22) PCT Filed: Apr. 23, 2021
(86) PCT No.: PCT/FR2021/050702

§ 371 Date: Oct. 20, 2022

(30) Foreign Application Priority Data
Apr. 24, 2020 (FR) 2004090

(51) Int. Cl. A01N 63/50; C07K 14/435; C07K 14/47; A01N 63/14

(52) U.S. Cl. CPC A01N 63/50; C07K 14/43563; C07K 14/4723; A01N 63/14

(57) ABSTRACT

The present invention relates to peptides for inhibiting the production of mycotoxins by fungi of the genus Fusarium and also to compositions comprising them and methods implementing them.

### FIELD OF THE INVENTION

The present invention relates to the phytosanitary field. In particular, it relates to means for controlling fungi of the genus Fusarium, notably biocontrol or phytosanitary control of contamination with category B trichothecene-type mycotoxins produced during fusariosis infestation.

### INTRODUCTION

Fusarium graminearum is a major causal agent of fusarium head blight of wheat and fusarium head blight of corn. These two fungal diseases have a substantial economic impact in many cereal-producing regions of the world. Furthermore, F. graminearum can produce mycotoxins, mainly category B trichothecenes or TCTBs (the major representatives of which are deoxynivalenol/DON and its acetylated forms 15 and 3 acetyldeoxynivalenol/15 and 3-ADON), which are toxic to humans and animals. Indeed, these toxins have acute toxicity proven to be the cause of serious and sometimes fatal food poisoning and very strongly suspected chronic toxicity (hematotoxicity and immunotoxicity). As thermostable molecules, TCTBs, accumulated in harvested grains, are not entirely eliminated during the manufacturing of cereal-based foodstuffs and animal feeds. The agronomic practices recommended for limiting the contamination of crops do not guarantee their compliance with the regulatory thresholds set by the EU (No. 1126/2007). It is thus urgent to implement new sustainable and environmentally friendly pre-harvest control strategies to limit the accumulation of TCTBs in grains.

Defensins are low molecular weight cyclic peptides having a conserved moiety known as the gamma-core, which are important for their antimicrobial activity. Among these defensins, tick defensins known as DefMT3 and DefMT6 have been described as having antibacterial and antifungal activity, notably against Fusarium culmorum and Fusarium graminearum (Tonk et al., 2015, Developmental and Comparative Immunology, 53, 358-365). In this article, the authors studied the activity of peptides having only the gamma base moiety of DefMT3 or DefMT6 and observed that these moieties showed enhanced antifungal activity compared to full-length defensin, in particular on spore germination and growth.

Targeting only the growth of the fungal species does not necessarily ensure lower mycotoxin levels. Indeed, the stress perceived by the fungus can be a factor for the stimulation of toxin production. Thus, it is essential to have control means specifically directed against the production of TCTBs.

There is thus still a need for a means for specifically controlling the production of category B trichothecene mycotoxins produced during fusariosis infestation.

### SUMMARY OF THE INVENTION

The present invention describes a method for reducing the production of mycotoxins by fungi of the genus Fusarium, in particular category B trichothecenes (TCTB).

Indeed, the inventors have identified the ability of certain peptides to inhibit the production of these mycotoxins. They observed that the peptide DefMT3, but also a peptide bearing the conserved gamma moiety of DefMT3 or variants thereof severely inhibit the production of DON (deoxynivalenol) and 15-ADON (15-acetyldeoxynivalenol) toxins by Fusarium graminearum. They showed that the linear peptide forms show more powerful inhibitory activity on mycotoxin production than the cyclic forms. In addition, the linear forms show fungal growth inhibitory activity that is almost absent for the cyclic forms. It was also shown that the cationic charge of the peptides was an important factor for their antifungal activity and their inhibitory activity on TCTBs biosynthesis. Finally, the inventors showed that the PEGylated forms showed improved antifungal activity.

Thus, the present invention relates to the use of a peptide for inhibiting the production of mycotoxins by fungi of the genus Fusarium on a plant, the peptide comprising a sequence chosen from

SEQ ID NO: 18
C/S-G/S-N/G-F/R/I-L/W/I-K/T-R/L/Q/T-T-C/S-I/T-
C/S-V/I/Y-K/M/R/T-K/N/T
SEQ ID NO: 19
C/S-G/S-N/G-F/I-L/I-K/T-R/Q/T-T-C/S-I/T-C/S-
V/Y-K/R/T-K/T
SEQ ID NO: 20
C/S-G/S-N/G-F/I-L/I-K/T-R/T-T-C/S-I/T-C/S-V/Y-
K/T-K/T,
and
SEQ ID NO: 21
C/S-G/S-N/G-F/I-L/I-K/R-R/K-T-C/S-I/T-C/S-V/Y-
K/R-K/R.

The invention also relates to a method for inhibiting the production of mycotoxins by fungi of the genus Fusarium on a plant, comprising placing the plant in contact with a composition comprising a peptide, the peptide comprising a sequence chosen from

SEQ ID NO: 18
C/S-G/S-N/G-F/R/I-L/W/I-K/T-R/L/Q/T-T-C/S-I/T-
C/S-V/I/Y-K/M/R/T-K/N/T
SEQ ID NO: 19
C/S-G/S-N/G-F/I-L/I-K/T-R/Q/T-T-C/S-I/T-C/S-V/Y
-K/R/T-K/T
SEQ ID NO: 20
C/S-G/S-N/G-F/I-L/I-K/T-R/T-T-C/S-I/T-C/S-V/Y-
K/T-K/T,
and
SEQ ID NO: 21
C/S-G/S-N/G-F/I-L/I-K/R-R/K-T-C/S-I/T-C/S-V/Y-
K/R-K/R.

In a particular aspect, the peptide comprises not more than 30 amino acids.

In a particular aspect, the peptide comprises a sequence chosen from the sequences SEQ ID Nos: 22 and 23, consists of a sequence chosen from the sequences SEQ ID Nos: 22 and 23 with the sequence possibly comprising 1, 2, 3, 4 or 5 substitutions, additions, deletions or mixtures thereof, or consists of a sequence chosen from the sequences SEQ ID Nos: 22 and 23. In an even more particular aspect, the peptide consists of the sequence SEQ ID NO: 22.

The peptide implemented in this use or this method may comprise, consist essentially of or consist of a sequence chosen from

SEQ ID NO: 2
C G N F L K R T C I C V K K
SEQ ID NO: 3
C S G 1 I K Q T C T C Y R K
SEQ ID NO: 6
C G N F L T R T C I C V K K
SEQ ID NO: 7
C G N F L K T T C I C V K K
SEQ ID NO: 8
C G N F L K R T C I C V T K
SEQ ID NO: 9
C G N F L K R T C I C V K T
SEQ ID NO: 10
C G N F L T T T C I C V T T.
SEQ ID NO: 11
S G N F L K R T S I S V K K
SEQ ID NO: 12
S S G I I K Q T S T S Y R K
SEQ ID NO: 13
S G N F L T R T S I S V K K
SEQ ID NO: 14
S G N F L K T T S I S V K K
SEQ ID NO: 15
S G N F L K R T S I S V T K
SEQ ID NO: 16
S G N F L K R T S I S V K T
and
SEQ ID NO: 17
S G N F L T T T S I S V T T.

It is also relates to a phytosanitary composition intended for treating a plant comprising a peptide, notably for inhibiting the production of mycotoxins by fungi of the genus Fusarium on the plant, the peptide comprising a sequence chosen from

SEQ ID NO: 18
C/S-G/S-N/G-F/R/I-L/W/I-K/T-R/L/Q/T-T-C/S-T/T-C/
S-V/I/Y-K/M/R/T-K/N/T
SEQ ID NO: 19
C/S-G/S-N/G-F/I-L/I-K/T-R/Q/T-T-C/S-I/T-C/S-V/
Y-K/R/T-K/T
SEQ ID NO: 20
C/S-G/S-N/G-F/I-L/I-K/T-R/T-T-C/S-I/T-C/S-V/
Y-K/T-K/T,
and
SEQ ID NO: 21
C/S-G/S-N/G-F/I-L/I-K/R-R/K-T-C/S-I/T-C/S-V/
Y-K/R-K/R,

the peptide comprising not more than 30 amino acids and

the peptide not being an unmodified peptide having the sequence

CGNFLKRTCICVKK SEQ ID NO: 2 or

CSGIIKQTCTCYRK SEQ ID NO: 3.

The peptide of the phytosanitary composition may comprise, consist essentially of or consist of a sequence chosen from

C G N F L K R T C I C V K K SEQ ID NO: 2, the peptide bearing a PEGylation, a C-terminal amidation, an N-terminal acetylation, a modified peptide bond, a D-form amino acid, a modified cysteine or a combination of these modifications;

C S G I I K Q T C T C Y R K SEQ ID NO: 3, the peptide bearing a PEGylation, a C-terminal amidation, an N-terminal acetylation, a modified peptide bond, a D-form amino acid, a modified cysteine or a combination of these modifications;

SEQ ID NO: 6
CGNFLTRTCICVKK
SEQ ID NO: 7
CGNFLKTTCICVKK
SEQ ID NO: 8
CGNFLKRTCICVTK
SEQ ID NO: 9
CGNFLKRTCICVKT
SEQ ID NO: 10
CGNFLTTTCICVTT
SEQ ID NO: 11
SGNFLKRTSISVKK
SEQ ID NO: 12
SSGIIKQTSTSYRK
SEQ ID NO: 13
SGNFLTRTSISVKK
SEQ ID NO: 14
SGNFLKTTSISVKK
SEQ ID NO: 15
SGNFLKRTSISVTK
SEQ ID NO: 16
SGNFLKRTSISVKT
and
SEQ ID NO: 17
SGNFLTTTSISVTT.

In particular embodiments of the use, method or composition, the peptide comprises not more than 20 amino acids.

In particular embodiments of the use, method or composition, the peptide is PEGylated.

In particular embodiments of the use, method or composition, the peptide does not comprise any disulfide bridges.

In particular embodiments of the use, method or composition, the mycotoxin is a category B trichothecene, preferably deoxynivalenol (DON) and/or acetylated deoxynivalenol (A-DON).

In particular embodiments of the use, method or composition, the fungus of the genus Fusarium is chosen from Fusarium culmorum, Fusarium graminearum, Fusarium tricinctum, Fusarium avenaceum, Fusarium poae, Fusarium sporotrichioides, Fusarium verticillioides, Fusarium proliferatum, Fusarium langsethiae, Fusarium oxysporum, Fusarium roseum, Fusarium arthrosporioides and Fusarium avenaceum, preferably Fusarium culmorum or Fusarium graminearum.

In particular embodiments of the use, method or composition, the plant is chosen from cereals, such as wheat (soft and durum), barley, corn, oat, triticale and rice, or fruits and vegetables such as tomato, melon, cucumber, zucchini, Jerusalem artichoke, bell pepper, potato, asparagus, sweet potato, celery, garlic, onion, cabbage, ginger, banana, cassava, vanilla, and fruit trees such as date palm, preferably cereals chosen from wheat (soft and durum), barley, corn, oat, triticale and rice.

### DETAILED DESCRIPTION OF THE INVENTION

### Definitions

In the present document, the terms “peptide”, “oligopeptide” and “polypeptide” are used interchangeably and refer to a chain of amino acids connected via peptide bonds, irrespective of the number of amino acid residues constituting said chain.

The peptide sequences defined herein are represented with the one-letter symbol as shown in Table 1.

TABLE 1

| | | |
|---|---|---|
| A | Ala | Alanine |
| R | Arg | Arginine |
| N | Asn | Asparagine |
| D | Asp | Aspartic acid |
| C | Cys | Cysteine |
| Q | Gln | Glutamine |
| E | Glu | Glutamic acid |
| G | Gly | Glycine |
| H | His | Histidine |
| I | Ile | Isoleucine |
| L | Leu | Leucine |
| K | Lys | Lysine |
| M | Met | Methionine |
| F | Phe | Phenylalanine |
| P | Pro | Proline |
| S | Ser | Serine |
| T | Thr | Threonine |
| W | Trp | Tryptophan |
| Y | Tyr | Tyrosine |
| V | Val | Valine |

As used herein, the term “amino acid” refers to the 20 naturally occurring standard amino acid residues (G, P, A, V, L, I, M, C, F, Y, W, H, K, R, Q, N, E, D, S and T), to rare naturally occurring amino acid residues (for example hydroxyproline, hydroxylysine, allohydroxylysine, 6-N-methylysine, N-ethylglycine, N-methylglycine, N-ethylasparagine, allo-isoleucine, N-methylisoleucine, N-methylvaline, aminobutyric acid) and to unnatural amino acids (for example norleucine, norvaline and cyclohexylalanine). Preferably, this term refers to the 20 naturally occurring standard amino acid residues (G, P, A, V, L, I, M, C, F, Y, W, H, K, R, Q, N, E, D, S and T).

The term “substitution” as used herein denotes the replacement of one amino acid residue with another one chosen from the 20 naturally occurring standard amino acid residues, rare naturally occurring amino acid residues and unnatural amino acids. Preferably, the term “substitution” refers to the replacement of one amino acid residue with another one chosen from the 20 naturally occurring standard amino acid residues (G, P, A, V, L, I, M, C, F, Y, W, H, K, R, Q, N, E, D, S and T). The substitution(s) may be conservative or non-conservative substitutions. As used herein, the term “conservative substitution” refers to a replacement of one amino acid residue with another one which has similar chemical or physical properties (size, charge or polarity). Examples of conservative substitutions are shown in the following tables.

TABLE 2

| Amino acid groups | Amino acid residues |
|---|---|
| Acidic | D and E |
| Basic | K, R and H |
| Uncharged hydrophilic | S, T, N and Q |
| Uncharged aliphatic | G, A, V, L and I |
| Uncharged nonpolar | C, M and P |
| Aromatic | F, Y and W |

TABLE 3

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE 4

| | |
|---|---|
| Residues with an alcohol group | S and T |
| Aliphatic residues | I, L, V and M |
| Residues with an aromatic ring | F, H, W and Y |
| Hydrophobic residues | A, C, F, G, H, I, L, M, R, T, V, W and Y |
| Negatively charged residues | D and E |
| Positively charged residues | K, R and H |
| Polar residues | C, D, E, H, K, N, Q, R, S and T |
| Small residues | A, C, D, G, N, P, S, T and V |
| Very small residues | A, G and S |
| Flexible residues | E, Q, T, K, S, G, P, D, E and R |

Unless otherwise specified or clearly contradicted by the context, the term “consists of” a particular sequence, should be understood as describing a peptide consisting of said sequence. The term “consists essentially of” means that the peptide consists of said sequence, but it may also comprise 1, 2, 3, 4, 5, 6, 7, 8, 9 or 10 substitutions, additions, deletions or a mixture thereof, preferably 1, 2, 3, 4 or 5 substitutions, additions, deletions or a mixture thereof, and in particular 1, 2 or 3 substitutions, additions, deletions or a mixture thereof. In particular, by the term “consisting essentially of” it can be envisaged that the peptide may comprise 1, 2, 3, 4, 5, 6, 7, 8, 9 or 10 additional amino acids at the N- and/or C terminal ends, preferably 1, 2, 3, 4 or 5 additional amino acids, and/or 1, 2 or 3 additional amino acids. Preferably, the number of substitutions, additions, deletions or a mixture thereof depends on the length of the sequence. For example, the percentage of substitutions, deletions, additions or a mixture thereof may be no more than 30%, preferably no more than 25%. As used herein, the term “substitution” refers to the exchange of a single amino acid for another one in a peptide sequence; the term “deletion” refers to the removal of a single amino acid from a peptide sequence; the terms “insertion” and “addition” are equivalent and refer to the addition of a single amino acid in a peptide sequence.

As used herein, the term “sequence identity” or “identity” refers to the number (%) of pairs (identical amino acid residues) at positions from an alignment of two polypeptide sequences. Sequence identity is determined by comparing the sequences when they are aligned so as to maximize the overlap and identity while at the same time minimizing the sequence breaks. In particular, sequence identity can be determined using any of a number of global or local alignment algorithms, depending on the length of the two sequences. Sequences of similar lengths are preferably aligned using global alignment algorithms (e.g. Needleman & Wunsch, J. Mol. Biol 48:443, 1970) which optimally align the sequences along the entire length, whereas sequences of substantially different lengths are preferably aligned using a local alignment algorithm, for example the Smith-Waterman algorithm (Smith and Waterman, Adv. Appl. Math. 2:482, 1981) or the Altschul algorithm (Altschul et al. (1997) Nucleic Acids Res. 25:3389-3402; Altschul et al. (2005) FEBS J. 272:5101-5109). Alignment for the purpose of determining the percentage of amino acid sequence identity can be performed via any method known to those skilled in the art, for example using software available on websites such as http://blast.ncbi.nlm. nih.gov/ or http://www.ebi.ac.uk/Tools/emboss/. A person skilled in the art can readily determine the appropriate parameters for measuring alignment. For purposes of the present invention, the amino acid sequence identity percentage values refer to values generated using the EMBOSS Needle pairwise sequence alignment program which creates an optimum global alignment of two sequences using the Needleman-Wunsch algorithm in which the parameters are the default parameters: Scoring matrix=BLOSUM62, Gap open=10, Gap extend=0.5, End gap penalty=false, End gap open=10 and End gap extend=0.5.

Peptides

The inventors identified peptides with inhibitory activity on the production of mycotoxins, in particular category B trichothecenes, by a fungus of the genus Fusarium.

The inhibitory activity on the production of mycotoxins, in particular of category B trichothecenes, can be measured via any technique known to those skilled in the art. For example, it can be measured as described in detail in the examples in the section “Mycotoxin production inhibition test” by measuring the amounts of DON and 15-ADON with the strain Fusarium graminearum CBS 185.32.

A peptide shows inhibitory activity on mycotoxin production when this production is reduced by 20, 30, 40, 50, 60, 70, 80, 90 or 100% compared to the production in the absence of the peptide.

The peptide with inhibitory activity on mycotoxin production by a fungus of the genus Fusarium comprises, consists essentially of or consists of

SEQ ID NO: 1
C/S-X₁-X₂-X₃-X₄-X₅-X₆-T-C/S-X₇-C/S-X₈-X₉-X₁₀

wherein,

C/S indicates cysteine (C) or serine (S), the cysteine may optionally be modified so as to prevent disulfide bridge formation;

X₁ is a small amino acid, for example chosen from G, S, A, V, T, D, N and P, in particular G, S or A, even more particularly G or S;

X₂ is a small amino acid, for example chosen from G, S, A, V, T, D, N and P, in particular G, S, A or N, even more particularly G or N;

X₃ is chosen from I, L, F, Y, W, M, R or K, in particular I, L, F or Y, even more particularly I or F;

X₄ is chosen from I, L, F, Y, W, or M, in particular I, L, W or F, even more particularly I or L;

X₅ is chosen from K, R, H, T, S, or Q, in particular K, R, S or T, even more particularly K or T, K, R or T, or K or R;

X₆ is chosen from R, K, L, I, Q or T, in particular R, K, Q or T, more even particularly Q, R or T, K, R or T, or K or R;

X₇ is chosen from I, L, M, T or S, in particular I, L or T, even more particularly I or T;

X₈ is chosen from V, I, L, Y, F, W or M, in particular V, I, L or Y, even more particularly V or Y;

X₉ is chosen from R, K, H, T, S, N, M or Q, in particular R, K, T or M, even more particularly R, K or T, preferably K or R; and

X₁₀ is chosen from S, T, Q, K, R, N or H, in particular K, R, N or T, even more particularly K or T, K, R or T, or K or R.

In a particular embodiment, the peptide comprises not more than 30 amino acids.

In a preferred embodiment, when the peptide comprises at least two cysteines, these cysteines are in the reduced form (SH), i.e. they do not form disulfide bridges together.

In another aspect, the peptide comprises serines when the sequence indicates C/S.

In another aspect, the peptide comprises one or more cysteines modified so as to prevent disulfide bridge formation. In particular, the cysteines may be alkylated on the thiol function, in particular methylated (S—CH₃), or by carboxamidomethylation (addition of a CH₂—CONH₂ group).

In a particular embodiment, all the cysteines of the peptide or all but one of the cysteines are modified or replaced with cysteines.

The peptide may comprise a sequence identity with the peptide of SEQ ID NO: 2 or 3 of at least 50, 60, 70, 80, 90 or 95%. Thus, the peptide may comprise 1, 2, 3, 4, or 5 substitutions relative to the peptide of SEQ ID NO: 2 or 3, notably in the positions of the residues chosen from X₁, X₂, X₃, X₄, X₅, X₆, X₇, X₈, X₉ and X₁₀.

In a particular embodiment, the peptide comprises, consists essentially of or consists of a sequence

SEQ ID NO: 18
C/S-G/S-N/G-F/R/I-L/W/I-K/T-R/L/Q/T-T-C/S-I/T-C/
S-V/I/Y-K/M/R/T-K/N/T
or
SEQ ID NO: 19
C/S-G/S-N/G-F/I-L/I-K/T-R/Q/T-T-C/S-I/T-C/S-V/
Y-K/R/T-K/T
or
SEQ ID NO: 20
C/S-G/S-N/G-F/I-L/I-K/T-R/T-T-C/S-I/T-C/S-V/
Y-K/T-K/T
or
SEQ ID NO: 21
C/S-G/S-N/G-F/I-L/I-K/R-R/K-T-C/S-I/T-C/S-V/
Y-K/R-K/R.

In a very particular embodiment, the peptide comprises, consists essentially of or consists of a sequence chosen from the group consisting of

SEQ ID NO: 2
TickCore3: C G N F L K R T C I C V K K
SEQ ID NO: 3
TickCore6: C S G I I K Q T C T C Y R K
SEQ ID NO: 6
TC3K6T: C G N F L T R T C I C V K K
SEQ ID NO: 7
TC3R7T: C G N F L K T T C I C V K K
SEQ ID NO: 8
TC3K13T: C G N F L K R T C I C V T K
SEQ ID NO: 9
TC3K14T: C G N F L K R T C I C V K T
and
SEQ ID NO: 10
TC3KRKKT: C G N F L T T T C I C V T T;

preferably chosen from the group consisting of:

SEQ ID NO: 2
TickCore3: C G N F L K R T C I C V K K
SEQ ID NO: 3
TickCore6: C S G I I K Q T C T C Y R K
SEQ ID NO: 6
TC3K6T: C G N F L T R T C I C V K K
SEQ ID NO: 7
TC3R7T: C G N F L K T T C I C V K K
SEQ ID NO: 8
TC3K13T: C G N F L K R T C I C V T K
and
SEQ ID NO: 9
TC3K14T: C G N F L K R T C I C V K T;

or from the group consisting of

SEQ ID NO: 2
TickCore3: C G N F L K R T C I C V K K
SEQ ID NO: 3
TickCore6: C S G I I K Q T C T C Y R K
SEQ ID NO: 7
TC3R7T: C G N F L K T T C I C V K K
SEQ ID NO: 8
TC3K13T: C G N F L K R T C I C V T K
and
SEQ ID NO: 9
TC3K14T: C G N F L K R T C I C V K T.

Optionally, the peptide may also comprise, consist essentially of or consist of a sequence chosen from the group consisting of

SEQ ID NO: 11
S G N F L K R T S I S V K K
SEQ ID NO: 12
S S G I I K Q T S T S Y R K
SEQ ID NO: 13
S G N F L T R T S I S V K K
SEQ ID NO: 14
S G N F L K T T S I S V K K
SEQ ID NO: 15
S G N F L K R T S I S V T K
SEQ ID NO: 16
S G N F L K R T S I S V K T
and
SEQ ID NO: 17
S G N F L T T T S I S V T T;

preferably chosen from the group consisting of:

SEQ ID NO: 11
S G N F L K R T S I S V K K
SEQ ID NO: 12
S S G I I K Q T S T S Y R K
SEQ ID NO: 14
S G N F L K T T S I S V K K
SEQ ID NO: 15
S G N F L K R T S I S V T K
and
SEQ ID NO: 16
S G N F L K R T S I S V K T.

In a very particular aspect, the peptide is not or does not comprise the sequence of a peptide chosen from

SEQ ID NO: 4
CGGRWKLTCICVRG
SEQ ID NO: 5
CNGPFNIVCSCY

Optionally, the peptide molecule may also comprise other peptide sequences on the N-terminal or C-terminal side. For example, the peptide may comprise, at the N-terminal or C-terminal, a tag that is useful for the purification or immobilization of the peptide. Such tags are well known to those skilled in the art and include, for example, histidine (His6), FLAG, HA (epitope derived from the influenza virus hemagglutinin), MYC (epitope derived from the human proto-oncoprotein MYC) or GST (glutathione-S-transferase) tags. Optionally, the peptide may comprise a protease cleavage site or a chemical agent for deleting this tag.

In a preferred embodiment, the peptide is less than 30 amino acids in length, preferably less than 25, 20, 19, 18, 17, 16 or 15 amino acids.

In a preferred embodiment, the peptide is not cyclic. Thus, it does not contain any covalent interactions between the side chains of two amino acids or between the N-terminal and C-terminal ends.

The peptide bond(s) of the peptide can be modified to make them resistant to proteolysis. For example, at least one peptide bond (—CO—NH—) may be replaced with a divalent bond chosen from (—CH2-NH—), (—NH—CO—), (—CH2-O—), (—CH2-S—), (—CH2-CH2-), (—CO-CH2-), (—CHOH-CH2-), (—N═N—) and (—CH═CH—). Optionally, all the peptide bonds may be replaced.

The peptide molecule may comprise either a carboxylic (—COO—) or amidated (—CONH2)C-terminal end. The C-terminal end may also be esterified. The peptide may also be optionally modified at its N-terminal end, for example with an acetyl radical. In a particular embodiment, the peptide may be PEGylated (polyethylene glycol), in part at its N-terminal end. For example, the peptide may comprise 1, 2, 3, 4, 5, 6, 7, 8, 9, 10 or more PEG groups.

The L and D isomers of amino acids are envisaged. Indeed, the D isomers are not sensitive to proteases and the present invention also comprises molecules comprising D-amino acids, notably molecules comprising only or predominantly D-amino acids. In a particular embodiment, L-amino acids are preferred. In another particular embodiment, the peptide may comprise only D-amino acids and may be in a retro-inverted configuration (same sequence but reversed).

In a particular aspect, the present invention relates to a novel peptide with inhibitory activity on mycotoxin production by a fungus of the genus Fusarium.

In particular, a novel peptide according to the present invention is a peptide as defined above but is not an unmodified peptide having the sequence

SEQ ID NO: 2
CGNFLKRTCICVKK
or
SEQ ID NO: 3
CSGIIKQTCTCYRK.

Thus, a novel peptide may be a peptide having the sequence SEQ ID NO: 2 or 3 which bears PEGylation, C-terminal amidation, N-terminal acetylation, a modified peptide bond, a D-form amino acid, a modified cysteine, or a combination of these modifications.

In a particular embodiment, the peptide comprises a sequence chosen from the sequences SEQ ID Nos: 22 and 23, consists of a sequence chosen from the sequences SEQ ID Nos: 22 and 23 with the sequence possibly comprising 1, 2, 3, 4 or 5 substitutions, additions, deletions or mixtures thereof, or consists of a sequence chosen from the sequences SEQ ID Nos: 22 and 23. In particular, the peptide may consist of the sequence SEQ ID NO: 22.

Compositions and Uses

The present invention relates to a composition comprising a novel peptide as described in the present patent application or a mixture thereof, the composition preferably being a phytosanitary composition, notably for controlling a fungus of the genus Fusarium, and more particularly for controlling the production of mycotoxins.

The present invention also relates to the use of a peptide as described in the present patent application or a mixture thereof for inhibiting the production of mycotoxins by fungi of the genus Fusarium on a plant.

Finally, the invention relates to a method for inhibiting the production of mycotoxins by fungi of the genus Fusarium on a plant, involving placing the plant in contact with a composition comprising a peptide as described in the present patent application or a mixture thereof.

The fungus of the genus Fusarium may notably be chosen from Fusarium culmorum, Fusarium graminearum, Fusarium tricinctum, Fusarium avenaceum, Fusarium poae, Fusarium sporotrichioides, Fusarium verticillioides, Fusarium proliferatum, Fusarium langsethiae, Fusarium oxysporum, Fusarium roseum, Fusarium arthrosporioides and Fusarium avenaceum, preferably from Fusarium culmorum, Fusarium graminearum, Fusarium tricinctum, Fusarium avenaceum, Fusarium poae, Fusarium sporotrichioides, Fusarium verticillioides, Fusarium proliferatum, Fusarium langsethiae and Fusarium oxysporum. In a particular aspect, the fungus is Fusarium culmorum or Fusarium graminearum.

The plants which this treatment concerns may be cereals, such as wheat (soft and durum), barley, corn, oat, triticale and rice, but also fruits and vegetables such as tomato, melon, cucumber, zucchini, Jerusalem artichoke, bell pepper, potato, asparagus, sweet potato, celery, garlic, onion, cabbage, ginger, banana, cassava and vanilla, and fruit trees such as date palm.

The mycotoxins are in particular category B trichothecenes. Notably, among the category B trichothecenes are deoxynivalenol (DON), acetylated deoxynivalenol (A-DON) including 3-acetyl-DON (3-ADON) and 15-acetyl-DON (15-ADON) type, nivalenol and fusarenone X.

In a particular embodiment, the phytosanitary composition or peptide is applied to the aerial part of the plant. Preferably, it is applied at the time of or before the flowering of the plant. Alternatively, it is applied at the time of grain formation.

In a particular aspect, the phytosanitary composition or peptide may be used at a concentration at which it has an inhibitory effect on the production of mycotoxins by fungi of the genus Fusarium and has little or no effect on the growth of fungi of the genus Fusarium. Indeed, the concentration will be chosen so as to obtain an optimum effect on mycotoxin production. Thus, the peptide concentration may be, for example, less than 100 μM, 75 μM, 50 μM, 25 μM or 15 μM.

In another particular aspect, the phytosanitary composition or peptide may be used at a concentration at which it has an effect on mycotoxin production by fungi of the genus Fusarium and also has an inhibitory effect on the growth of fungi of the genus Fusarium. In this context, the peptide is preferably a novel peptide as defined in the present patent application.

### FIGURES

FIG. 1: Antifungal and antimycotoxin activity of TickCore3, native and oxidized forms. After 10 days of incubation in media supplemented or not supplemented with TickCore3 (native form) or TickCore3Ox (oxidized forms), the mycelia of F. graminearum were separated out and weighed. The mycelium weight values are given in grams (g) (FIG. 1a). The TCTB mycotoxins, DON (FIG. 1b) and 15-ADON (FIG. 1c), were quantified and expressed in μg of mycotoxins per ml of medium, then divided by the weight of mycelium and expressed in μg of TCTB mycotoxins per g of dry mycelium biomass (μg/g).

FIG. 2: Antifungal and antimycotoxin activity of linear and cyclic TickCore3 peptides. After 10 days of incubation in media supplemented or not supplemented with TickCore3 CH3-1 Ox, TickCore3 CH3-2 Ox, TickCore3 CH3-3 Ox, or TickCore3 CH3-123, the F. graminearum mycelia were separated out and weighed. The mycelium weight values are given in grams (g) (FIG. 2a). The TCTB mycotoxins, DON (FIG. 2b) and 15-ADON (FIG. 2c), were quantified in μg of mycotoxins per ml of medium, then divided by the weight of mycelium and expressed in μg of TCTB mycotoxins per g of dry mycelium biomass (μg/g).

FIG. 3: Antifungal and “antimycotoxin” activity of Tickcore3, native form or substituted forms in which the basic amino acids (K in position 6, 13 and 14, and R in position 7) have been replaced with T, an uncharged amino acid. After 10 days of incubation in media supplemented or not supplemented with TickCore3, TickCore3-K6T, TickCore3-R7T, TickCore3-K13T, and TickCore3-K14T, the F. graminearum mycelia were separated out and weighed. The mycelium weight values are given in grams (g) (FIG. 3a). The TCTB mycotoxins, DON (FIG. 3b) and 15-ADON (FIG. 3c), were quantified in μg of mycotoxins per ml of medium, then divided by the weight of mycelium and expressed in μg of TCTB mycotoxins per g of dry mycelium biomass (μg/g). Three different concentrations of peptide (12.5 μM, 25 μM and 50 μM) were tested.

FIG. 4: Antifungal and antimycotoxin activity of TickCore3 peptide and of a PEGylated version TickCore3-PEG. After 10 days of incubation in media supplemented or not supplemented with TickCore3 or TickCore3-PEG, the F. graminearum mycelia were separated out by centrifugation and weighed. The mycelium weight values are given in grams (g) (FIG. 4a). The TCTB mycotoxins, DON (FIG. 4b) and 15-ADON (FIG. 4c), were quantified in μg of mycotoxins per ml of medium, then divided by the weight of mycelium and expressed in μg of TCTB mycotoxins per g of dry mycelium biomass (μg/g).

FIG. 5: Antimycotoxin activity of the tick defensin DefMT3. After 10 days of incubation in media supplemented or not supplemented with DefMT3, the TCTB mycotoxins, DON (FIG. 5A) and 15-ADON (FIG. 5B), were quantified in μg of mycotoxin per ml of medium, divided by the mycelium weight, and expressed in μg of TCTB mycotoxin per g of dry mycelium biomass (μg/g). Two concentrations of DefMT3 were tested: 25 and 50 μM.

### EXAMPLES

The inventors have shown that the conserved linear gamma core moiety, with reduced cysteine (Cys) residues, of the tick defensin DefMT3, referred to hereinbelow as “TickCore3”, inhibits the production of category B trichothecenes (TCTB) by F. graminearum. Alkylation of all the Cys residues ofTickCore3 with methyl groups (TickCore3-CH3) had little effect on the inhibitory effect affecting TCTB production. Conversely, oxidation of all the Cys residues of TickCore3 strongly decreased the inhibitory effect on TCTB production. A major loss of inhibitory function was observed when the specific Cys4-Cys6 and Cys4-Cys5 disulfide bridges were formed. The inventors also showed that the cationic charge of the peptide was an important factor in its biological activity. Replacement of the positively charged amino acids with a neutral residue occasionally led to a very significant reduction in antifungal activity and in inhibition of TCTB production.

Results

TickCore3 is a Highly Effective Inhibitor of TCTB Production by F. graminearum

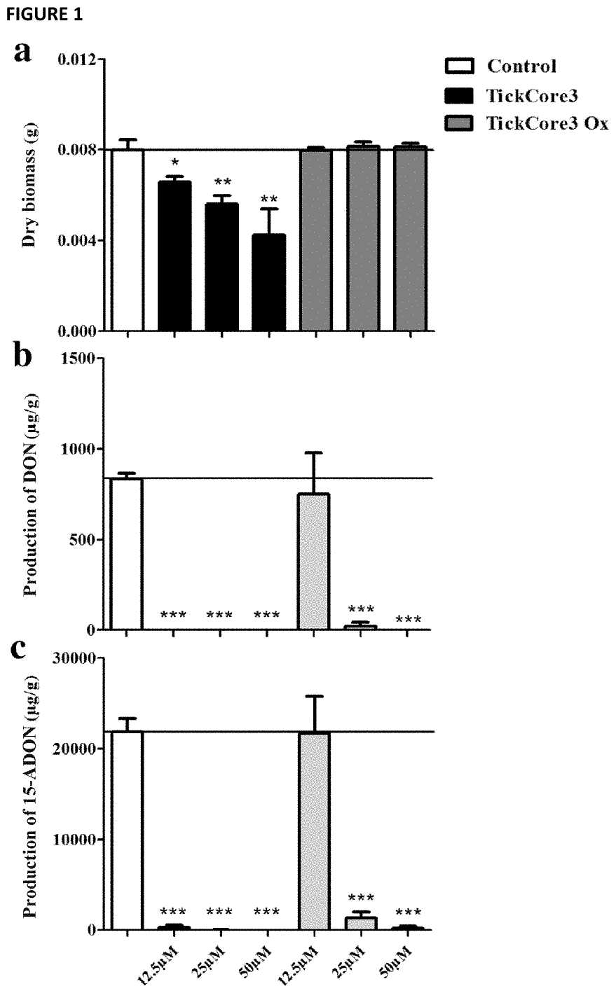

FIG. 1 shows the results obtained after 10 days of culturing the strain F. graminearum CBS 185.32 in MS medium supplemented with TickCore3 and its oxidized variant (TickCore3 Ox) at three different concentrations (12.5, 25 and 50 μM). Untreated culture media (control) were not supplemented with any of the TickCore3 peptides. Although TickCore3 Ox had no effect on the dry fungal biomass, TickCore3 significantly inhibited fungal growth at all the concentrations tested compared to the control condition (FIG. 1a). The inhibitory effect on fungal growth of TickCore3 was dose-dependent since increasing the concentration of the peptide had a proportional effect on the amount of dry biomass. As regards mycotoxin production, 15-ADON was the main TCTB produced in the liquid media by the strain studied. Under the control conditions, 15-ADON production (mean 21867 SD±3259 μg/g dry biomass) was 26 times higher than that of DON (835±68 μg/g). Supplementation with TickCore3 led to non-detectable levels of DON (FIG. 1b) and 15-ADON (FIG. 1c) for all the concentrations tested. However, residual levels of 15-ADON were observed with the 12.5 μM concentration (FIG. 1c). In contrast, a 12.5 μM concentration of TickCore3 Ox did not significantly reduce DON (FIG. 1b) or 15-ADON (FIG. 1c) production.

Cyclization of TickCore3 Reduces the Antifungal Activity

The inventors hypothesized that cycle formation in TickCore3 may have effects on inhibiting TCTB production. In order to test this hypothesis, TickCore3 derived peptides were synthesized by alkylating each Cys residue individually with a methyl group (CH3), followed by an oxidation protocol which generated peptides with disulfide bridges established between Cys5-Cys6 (TickCore3 CH3-1 Ox), Cys4-Cys6 (TickCore3 CH3-2 Ox) and Cys4-Cys5 (TickCore3 CH3-3 Ox).

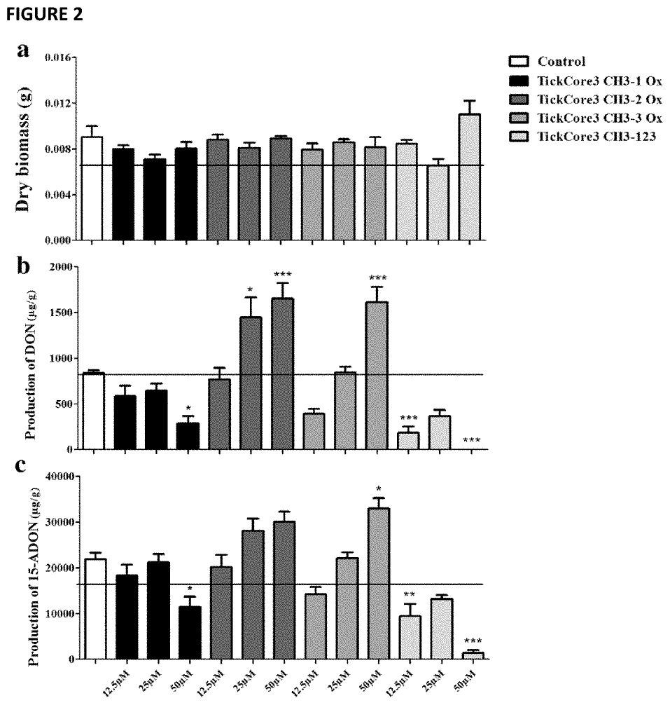

TickCore3-CH3-1 Ox, TickCore3-CH3-2 Ox, and TickCore3-CH3-3 Ox had no effect on fungal growth at any of the concentrations tested, in comparison with the control (FIG. 2a). The mycotoxin production by F. graminearum was then measured in media supplemented with TickCore3-CH3-1 Ox, TickCore3-CH3-2 Ox and TickCore3-CH3-3 Ox at three different concentrations: 12.5, 25 and 50 μM (FIG. 2b). Increasing the concentrations of TickCore3-CH3-2 Ox and TickCore3-CH3-3 Ox resulted in a significant increase in DON (FIG. 2b) and 15-ADON (FIG. 2c) production, except for the fungi exposed to TickCore3-CH3-2 Ox for which 15-ADON production did not change significantly. Moreover, treatment with TickCore3-CH3-1 Ox at 50 μM resulted in a significant reduction in DON and 15-ADON levels (FIG. 2bc). An additional peptide, TickCore3-CH3-CH3-123, with all the Cys residues alkylated with methyl groups, was synthesized. F. graminearum exposed to TickCore3-CH3-123 produced significantly less DON and 15-ADON in comparison with the control conditions.

The Cationic Charge is a Key Element of the Antifungal and “Antimycotoxin” Activity of TickCore3.

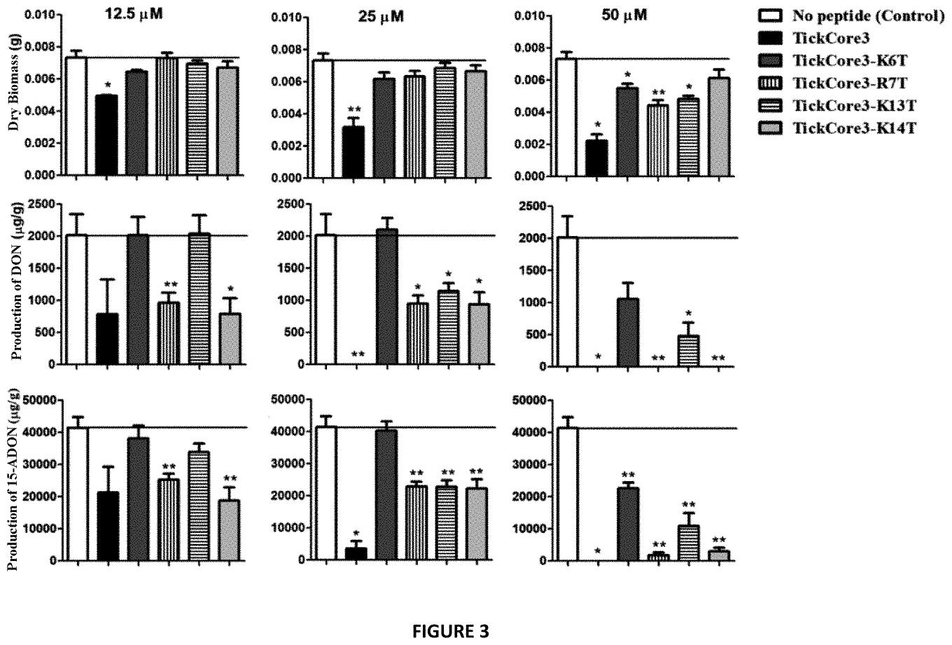

The inventors hypothesized that the cationic charge of TickCore 3 was an important element of its biological activity. In order to test this hypothesis, substituted TickCore3 peptides were synthesized by replacing the “basic” amino acids (arginine and lysine, R and K) with an uncharged residue, threonine (T). The following peptides were thus generated: TickCore3-K6T (replacement of lysine at position 6 with a threonine residue), TickCore3-R7T (replacement of arginine at position 7 with a threonine residue), Tickcore3-K13T (replacement of lysine at position 13 with a threonine residue), and TickCore3-K14T (replacement of lysine at position 14 with a threonine residue). For all the substituted peptides, the antifungal activity was reduced compared to that of the native peptide (FIG. 3a). The substituted forms (TickCore3-K13T, TickCore3-K14T and TickCore3-R7T) showed strong antifungal activity only at 50 μM, whereas TickCore3-K6T had no antifungal activity below the concentration at 50 μM. The mycotoxin production by F. graminearum was then measured in media supplemented with TickCore3, TickCore3-K13T, TickCore3-K6T, TickCore3-K14T and TickCore3-R7T at three different concentrations: 12.5, 25 and 50 μM (FIGS. 3b and 3c). For all the substituted peptides, whether with respect to DON (FIG. 3b) or 15-ADON (FIG. 3c) production, the ability to inhibit mycotoxin production is lower than that of the native form TickCore3. This reduction in the ability to inhibit TCTB production is particularly pronounced for TickCore3-K6T and to a lesser extent for TickCore3-K13T, suggesting that the lysine amino acids at positions 13 and 6 play an important role in the “antimycotoxin” activity.

PEGylation of TickCore3 Increases the Antifungal Activity

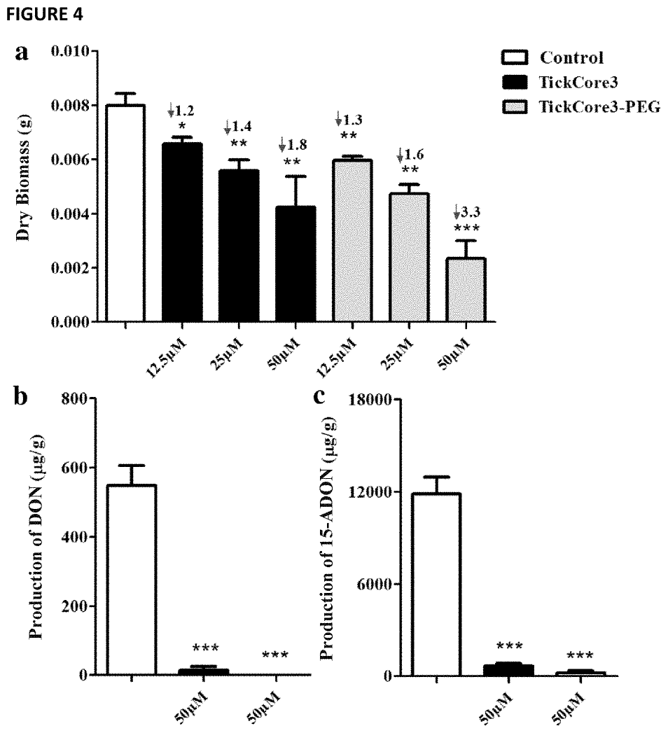

The peptide TickCore3-PEG was synthesized in the same way as TickCore3, except that two PEG units were sequentially attached to the N-terminal end of the peptide. Compared with TickCore3, TickCore3-PEG at 50 μM showed a greater antifungal effect with a 3.3-fold decrease in fungal growth (FIG. 4a). Furthermore, TickCore3-PEG is also considered to have improved inhibitory activity on mycotoxin production (FIG. 4bc).

The Defensin DefMT3 is Capable of Inhibiting Mycotoxin Production.

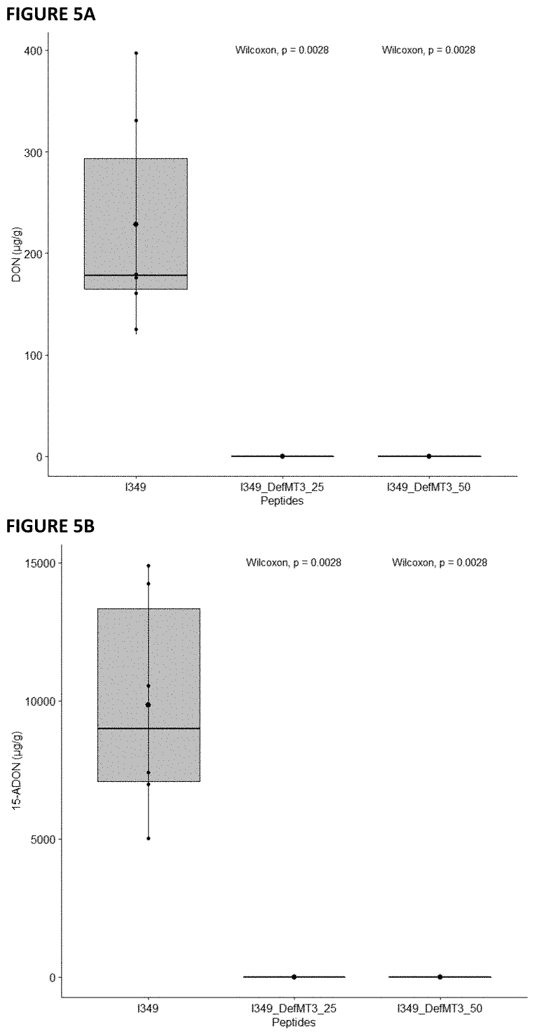

The peptide DefMT3 was synthesized in the same manner as TickCore3. This peptide shows inhibitory activity on production of the mycotoxins DON and 15-ADON (FIGS. 5a and 5b).

Materials and Methods

Synthesis of the Gamma Core of TickCore3

TickCore3 (CGNFLKRTCRTCICVKKK, SEQ ID NO: 2) is the conserved gamma core moiety of DefMT3 (GenBank accession number: JAA71488; Tonk et aL., 2015). The peptide synthesis was commissioned from Pepmic (Suzhou, China), which used solid phase peptide synthesis (SPPS) to obtain peptides with a high degree of purity as described previously in Cabezas-Cruz et al., 2016, (Front. Microbiol., 7, 1682). Briefly, the peptide synthesis was performed using 2-chlorotrityl chloride resin as a solid support, and 9-fluorenyl-methyloxy-carbonyl (Fmoc) labile base as a protecting group. The amino acids were protected as follows: Fmoc-Cys(Trt)-OH, Fmoc-Gly-OH, Fmoc-Asn(Trt)-OH, Fmoc-Phe-OH, Fmoc-Leu-OH, Fmoc-Lys(Boc)-OH, Fmoc-Arg(Pbf)-OH, Fmoc-Thr(tBu)-OH, Fmoc-Ile-OH and Fmoc-Val-OH. All the peptide sequences were synthesized according to SPPS principles. The peptides were purified by high performance liquid chromatography (HPLC) according to the standard peptide chemical coupling protocols.

The peptides purified by reverse-phase HPLC were then optionally oxidized using a refolding buffer containing 1 M urea, 100 mM Tris (pH 8.0), 1.5 mM oxidized glutathione, 0.75 mM reduced glutathione, and 10 mM methionine. Oxidation of the Cys residues was confirmed by the Ellman reaction and disulfide bond formation was characterized by electrospray mass spectroscopy (ESI-MS) using an LCMS-2020 mass spectrometer (Shimadzu, Kyoto, Kyoto Prefecture, Japan). The sequence composition was also verified by ESI-MS using an LCMS-2020 mass spectrometer (Shimadzu, Kyoto, Kyoto Prefecture, Japan). The peptides TickCore3-CH3-123 (C(CH3)GNFLKRTC(CH3)IC(CH3)VKK), TickCore3-CH3-1 (C(CH3)GNFLKRTCRICVKK), TickCore3-CH3-2 (CGNFLKRTC(CH3)ICVKK) and TickCore3-CH3-3 (CGNFLKRTCIC(CH3)VKK) were synthesized as described previously, except that Fmoc-Cys(Me)-OH was added in replacement for Fmoc-Cys(Trt)-OH. Alkylation of the sulfur (S) of the thiol groups of all the Cys residues prevents the cyclization of these residues (i.e. no disulfide bridges can be formed). Conversely, alkylation of the S of the thiol group of a single Cys residue, followed by its oxidation, directs the cyclization of the reduced Cys residues (i.e. those which have not been alkylated).

The peptide TickCore3-PEG was synthesized as for TickCore3, but two PEG units were sequentially attached to the N-terminal end of the peptide using protected Fmoc-NH-PEG2-CH2CH2COOH groups.

The peptide DefMT3 having the sequence GGYYCPFRQDKCHRHCRSFGRKAGYCGNFLKRTCICVKK (SEQ ID NO: 22) was synthesized according to a protocol identical to that of TickCore3 by the company Pepmic (Suzhou, China).

Test of Inhibition of Mycotoxin Production

Fusarium Strain and Culture Conditions

The strain F. graminearum CBS 185.32 which produces DON and 15-ADON (Centraal bureau voor Schimmelkulturen, The Netherlands) was used throughout this study. The fungal culture was maintained at 4° C. on potato dextrose agar (PDA) (Difco, Le Ponts de Claix, France) in slanted tubes under mineral oil. For the inoculum realization, the strain was cultured at 25° C. in the dark on PDA slants for 7 days and the spore suspension was prepared by adding 6 mL of sterile distilled water to the PDA slant with gentle agitation.

Liquid culture experiments were performed using 24-well static plates. Each well containing 2 ml of a synthetic medium (SM medium) which promotes rapid induction of TCTB production, prepared as previously published (Boutigny et aL., 2009, Mycol. Res., 113, 746), supplemented or not supplemented with the peptide, was inoculated with 2×10⁴ spores/mL. The fungal liquid cultures were incubated at 25° C. in the dark for 10 days. After incubation, the mycelia were recovered by centrifugation and the fungal biomass was measured by weighing the mycelia after 48 hours of freeze-drying (Flexi-Dry®, OErlikon Leybold, Germany). The culture media were stored at −20° C. until the time of TCTB analysis. All the peptides were tested at three concentrations: 12.5, 25 and 50 μM. Five repetitions were performed for each condition. DefMT3 was tested at 25 and 50 μM. Suitable controls using peptide-free control media and non-inoculated control media were included.

TCTB Extraction and Analysis

A 1.5-mL sample of culture medium was extracted with 3 mL of ethyl acetate. A volume of 2.5 mL of the organic phase was evaporated to dryness at 45° C. under a stream of nitrogen. The dried samples were dissolved in 200 μL of methanol/water (1/1, v/v) and filtered through a 0.2 am filter before analysis. The TCTBs were quantified by HPLC-DAD using an Agilent Technologies 1100 series liquid chromatograph equipped with an automatic sampling system, an Agilent diode array detector (DAD) and ChemStation chromatography management software (Agilent, France). Separation was performed on a Kinetex XB-C18 100 Å column (4.6×150 mm, 2.6 μm) (Phenomenex, France) maintained at 45° C. The mobile phase consisted of water acidified with orthophosphoric acid at pH 2.6 (solvent A) and acetonitrile (solvent B). The flow rate was maintained at 1 mL·min⁻¹. The injection volume was 5 μL. The TCTBs were separated out using an elution gradient: 7 to 30% B in 10 min, 30-90% B in 5 min, 90% B for 5 min, 90 to 7% B in 2 min, 7% B for 5 min. The UV-Vis spectra were recorded from 190 to 400 nm and the peak areas were measured at 230 nm. Quantification was performed by external calibration with standard solutions (Romer Labs, Austria). The toxin yields were expressed in μg·g⁻¹ of dry biomass.

Statistical Analyses

All the values presented are means±standard deviation including four biological replications. Since the data did not follow a normal distribution (Shapiro-Wilk normality test), Kruskal-Wallis one-way analysis was used, with comparisons of means performed using the Connover-Inman test. Statistical analysis was performed with the XLSTAT 2017 software (Addinsoft, Rennes, France). The statistical significance level p=0.05 was used throughout the study.

1-14. (canceled)

15. A method for inhibiting the production of mycotoxins by fungi of the genus Fusarium on a plant, involving placing the plant in contact with a composition comprising a peptide, the peptide comprising a sequence selected from:
(SEQ ID NO: 18)
C/S-G/S-N/G-F/R/I-L/W/I-K/T-R/L/Q/T-T-C/S-I/T-C/
S-V/I/Y-K/M/R/T-K/N/T;
(SEQ ID NO: 19)
C/S-G/S-N/G-F/I-L/I-K/T-R/Q/T-T-C/S-I/T-C/S-V/Y-K/
R/T-K/T;
(SEQ ID NO: 20)
C/S-G/S-N/G-F/I-L/I-K/T-R/T-T-C/S-I/T-C/S-V/Y-K/
T-K/T;
and
(SEQ ID NO: 21)
C/S-G/S-N/G-F/I-L/I-K/R-R/K-T-C/S-ET-C/S-V/Y-K/
R-K/R.

16. The method according to claim 15, wherein the peptide comprises not more than 20 or 30 amino acids.

17. The method according to claim 15, wherein the peptide comprises a sequence selected from the following sequences with the sequence optionally comprising 1, 2, 3, 4 or 5 substitutions, additions, deletions or a mixture thereof:
(SEQ ID NO: 2)
C G N F L K R T C I C V K K;
(SEQ ID NO: 3)
C S G I I K Q T C T C Y R K;
(SEQ ID NO: 6)
C G N F L T R T C I C V K K;
(SEQ ID NO: 7)
C G N F L K T T C I C V K K;
(SEQ ID NO: 8)
C G N F L K R T C I C V T K;
(SEQ ID NO: 9)
C G N F L K R T C I C V K T;
(SEQ ID NO: 10)
C G N F L T T T C I C V T T;
(SEQ ID NO: 11)
S G N F L K R T S I S V K K;
(SEQ ID NO: 12)
S S G I I K Q T S T S Y R K;
(SEQ ID NO: 13)
S G N F L T R T S I S V K K;
(SEQ ID NO: 14)
S G N F L K T T S I S V K K;
(SEQ ID NO: 15)
S G N F L K R T S I S V T K;
(SEQ ID NO: 16)
S G N F L K R T S I S V K T;
and
(SEQ ID NO: 17)
S G N F L T T T S I S V T T.

18. The method according to claim 15, wherein the peptide is PEGylated.

19. The method according to claim 15, wherein the peptide does not comprise any disulfide bridges.

20. The method according to claim 15, wherein the peptide comprises a sequence selected from SEQ ID NOs: 22 and 23, the sequence optionally comprising 1, 2, 3, 4 or substitutions, additions, deletions or mixtures thereof.

21. The method according to claim 15, wherein the peptide consists of SEQ ID NO: 22 or 23.

22. The method according to claim 15, wherein the mycotoxin is a category B trichothecene, deoxynivalenol (DON) and/or acetylated deoxynivalenol (A-DON).

23. The method according to claim 15, wherein the fungus of the genus Fusarium is selected from Fusarium culmorum, Fusarium graminearum, Fusarium tricinctum, Fusarium avenaceum, Fusarium poae, Fusarium sporotrichioides, Fusarium verticilioides, Fusarium proliferatum, Fusarium langsethiae, Fusarium oxysporum, Fusarium roseum, Fusarium arthrosporioides and Fusarium avenaceum.

24. The method according to claim 15, wherein the plant is selected from cereals, wheat, barley, corn, oat, triticale, rice, fruits, and vegetables.

25. The method according to claim 24, wherein the plant is selected from tomato, melon, cucumber, zucchini, Jerusalem artichoke, bell pepper, potato, asparagus, sweet potato, celery, garlic, onion, cabbage, ginger, banana, manioc and vanilla and date palm.

26. A phytosanitary composition intended for treating a plant comprising a peptide or for inhibiting the production of mycotoxins by fungi of the genus Fusarium on the plant, the peptide comprising a sequence selected from:
(SEQ ID NO: 18)
C/S-G/S-N/G-F/R/I-L/W/I-K/T-R/L/Q/T-T-C/S-I/T-C/
S-V/I/Y-K/M/R/T-K/N/T;
(SEQ ID NO: 19)
C/S-G/S-N/G-F/I-L/I-K/T-R/Q/T-T-C/S-I/T-C/S-V/
Y-K/R/T-K/T;
(SEQ ID NO: 20)
C/S-G/S-N/G-F/I-L/I-K/T-R/T-T-C/S-I/T-C/S-V/Y-K/
T-K/T;
and
(SEQ ID NO: 21)
C/S-G/S-N/G-F/I-L/I-K/R-R/K-T-C/S-ET-C/S-V/Y-K/
R-K/R,
the peptide comprising not more than 30 amino acids and the peptide not being an unmodified peptide having the sequence CGNFLKRTCICVKK (SEQ ID NO: 2) or CSGIIKQTCTCYRK (SEQ ID NO: 3).

27. The composition according to claim 26, wherein the peptide comprises a sequence selected from:
C G N F L K R T C I C V K K (SEQ ID NO: 2), the peptide bearing a PEGylation, a C-terminal amidation, an N-terminal acetylation, a modified peptide bond, a D-form amino acid, a modified cysteine or a combination of these modifications;
C S G I I K Q T C T C Y R K (SEQ ID NO: 3), the peptide bearing a PEGylation, a C-terminal amidation, an N-terminal acetylation, a modified peptide bond, a D-form amino acid, a modified cysteine or a combination of these modifications;
(SEQ ID NO: 6)
C G N F L T R T C I C V K K;
(SEQ ID NO: 7)
C G N F L K T T C I C V K K;
(SEQ ID NO: 8)
C G N F L K R T C I C V T K;
(SEQ ID NO: 9)
C G N F L K R T C I C V K T;
(SEQ ID NO: 10)
C G N F L T T T C I C V T T;
(SEQ ID NO: 11)
S G N F L K R T S I S V K K;
(SEQ ID NO: 12)
S S G I I K Q T S T S Y R K ;
(SEQ ID NO: 13)
S G N F L T R T S I S V K K;
(SEQ ID NO: 14)
S G N F L K T T S I S V K K;
(SEQ ID NO: 15)
S G N F L K R T S I S V T K;
(SEQ ID NO: 16)
S G N F L K R T S I S V K T;
and
(SEQ ID NO: 17)
S G N F L T T T S I S V T T.

28. The composition according to claim 26, wherein the peptide comprises not more than 20 or 30 amino acids.

29. The composition according to claim 26, wherein the peptide is PEGylated.

30. The composition according to claim 26, wherein the peptide does not comprise any disulfide bridges.

31. The composition according to claim 26, wherein the mycotoxin is a category B trichothecene, deoxynivalenol (DON) and/or acetylated deoxynivalenol (A-DON).

32. The composition according to claim 26, wherein the fungus of the genus Fusarium is selected from Fusarium culmorum, Fusarium graminearum, Fusarium tricinctum, Fusarium avenaceum, Fusarium poae, Fusarium sporotrichioides, Fusarium verticilioides, Fusarium proliferatum, Fusarium langsethiae, Fusarium oxysporum, Fusarium roseum, Fusarium arthrosporioides and Fusarium avenaceum.

33. The composition according to claim 26, wherein the plant is selected from cereals, wheat, barley, corn, oat, triticale, rice, fruits, and vegetables or selected from tomato, melon, cucumber, zucchini, Jerusalem artichoke, bell pepper, potato, asparagus, sweet potato, celery, garlic, onion, cabbage, ginger, banana, manioc and vanilla and date palm.


SEQUENCE LISTING (23 sequences)

SEQ ID NO: 1 - protein, 14 aa, artificial
  peptide
  MISC_FEATURE (1)..(1): Xaa is C or S
  MISC_FEATURE (2)..(3): Xaa is a small amino acid
  MISC_FEATURE (4)..(4): Xaa is I, L, F, Y, W, M, R or K
  MISC_FEATURE (5)..(5): Xaa is I, L, F, Y, W, or M
  MISC_FEATURE (6)..(6): Xaa is K, R, H, T, S, or Q
  MISC_FEATURE (7)..(7): Xaa is R, K, L, I, Q or T
  MISC_FEATURE (9)..(9): Xaa is C or S
  MISC_FEATURE (10)..(10): Xaa is I, L, M, T or S
  MISC_FEATURE (11)..(11): Xaa is C or S
  MISC_FEATURE (12)..(12): Xaa is V, I, L, Y, F, W or M
  MISC_FEATURE (13)..(13): Xaa is S, T, Q, K, R, N or H
  misc_feature (14)..(14): Xaa can be any naturally occurring amino acid
XXXXXXXTXXXXXX

SEQ ID NO: 2 - protein, 14 aa, artificial
  peptide
CGNFLKRTCICVKK

SEQ ID NO: 3 - protein, 14 aa, artificial
  peptide
CSGIIKQTCTCYRK

SEQ ID NO: 4 - protein, 14 aa, artificial
  peptide
CGGRWKLTCICVRG

SEQ ID NO: 5 - protein, 12 aa, artificial
  peptide
CNGPFNIVCSCY

SEQ ID NO: 6 - protein, 14 aa, artificial
  peptide
CGNFLTRTCICVKK

SEQ ID NO: 7 - protein, 14 aa, artificial
  peptide
CGNFLKTTCICVKK

SEQ ID NO: 8 - protein, 14 aa, artificial
  peptide
CGNFLKRTCICVTK

SEQ ID NO: 9 - protein, 14 aa, artificial
  peptide
CGNFLKRTCICVKT

SEQ ID NO: 10 - protein, 14 aa, artificial
  peptide
CGNFLTTTCICVTT

SEQ ID NO: 11 - protein, 14 aa, artificial
  peptide
SGNFLKRTSISVKK

SEQ ID NO: 12 - protein, 14 aa, artificial
  peptide
SSGIIKQTSTSYRK

SEQ ID NO: 13 - protein, 14 aa, artificial
  peptide
SGNFLTRTSISVKK

SEQ ID NO: 14 - protein, 14 aa, artificial
  peptide
SGNFLKTTSISVKK

SEQ ID NO: 15 - protein, 14 aa, artificial
  peptide
SGNFLKRTSISVTK

SEQ ID NO: 16 - protein, 14 aa, artificial
  peptide
SGNFLKRTSISVKT

SEQ ID NO: 17 - protein, 14 aa, artificial
  peptide
SGNFLTTTSISVTT

SEQ ID NO: 18 - protein, 14 aa, artificial
  peptide
  MISC_FEATURE (1)..(1): Xaa is C or S
  MISC_FEATURE (2)..(2): Xaa is G or S
  MISC_FEATURE (3)..(3): Xaa is N or G
  MISC_FEATURE (4)..(4): Xaa is F, R or I
  MISC_FEATURE (5)..(5): Xaa is L, W or I
  MISC_FEATURE (6)..(6): Xaa is K or T
  MISC_FEATURE (7)..(7): Xaa is R, L, Q or T
  MISC_FEATURE (9)..(9): Xaa is C or S
  MISC_FEATURE (10)..(10): Xaa is I or T
  MISC_FEATURE (11)..(11): Xaa is C or S
  MISC_FEATURE (12)..(12): Xaa is V, I or Y
  MISC_FEATURE (13)..(13): Xaa is K, M, R or T
  MISC_FEATURE (14)..(14): Xaa is K, N or T
XXXXXXXTXXXXXX

SEQ ID NO: 19 - protein, 14 aa, artificial
  peptide
  MISC_FEATURE (1)..(1): Xaa is C or S
  MISC_FEATURE (2)..(2): Xaa is G or S
  MISC_FEATURE (3)..(3): Xaa is N or G
  MISC_FEATURE (4)..(4): Xaa is F or I
  MISC_FEATURE (5)..(5): Xaa is L or I
  MISC_FEATURE (6)..(6): Xaa is K or T
  MISC_FEATURE (7)..(7): Xaa is R, Q or T
  MISC_FEATURE (9)..(9): Xaa is C or S
  MISC_FEATURE (10)..(10): Xaa is I or T
  MISC_FEATURE (11)..(11): Xaa is C or S
  MISC_FEATURE (12)..(12): Xaa is V or Y
  MISC_FEATURE (13)..(13): Xaa is K, R or T
  MISC_FEATURE (14)..(14): Xaa is K or T
XXXXXXXTXXXXXX

SEQ ID NO: 20 - protein, 14 aa, artificial
  peptide
  MISC_FEATURE (1)..(1): Xaa is C or S
  MISC_FEATURE (2)..(2): Xaa is G or S
  MISC_FEATURE (3)..(3): Xaa is N or G
  MISC_FEATURE (4)..(4): Xaa is F or I
  MISC_FEATURE (5)..(5): Xaa is L or I
  MISC_FEATURE (6)..(6): Xaa is K or T
  MISC_FEATURE (7)..(7): Xaa is R or T
  MISC_FEATURE (9)..(9): Xaa is C or S
  MISC_FEATURE (10)..(10): Xaa is I or T
  MISC_FEATURE (11)..(11): Xaa is C or S
  MISC_FEATURE (12)..(12): Xaa is V or Y
  MISC_FEATURE (13)..(13): Xaa is K or T
  MISC_FEATURE (14)..(14): Xaa is K or T
XXXXXXXTXXXXXX

SEQ ID NO: 21 - protein, 14 aa, artificial
  peptide
  MISC_FEATURE (1)..(1): Xaa is C or S
  MISC_FEATURE (2)..(2): Xaa is G or S
  MISC_FEATURE (3)..(3): Xaa is G or N
  MISC_FEATURE (4)..(4): Xaa is F or I
  MISC_FEATURE (5)..(5): Xaa is L or I
  MISC_FEATURE (6)..(7): Xaa is K or R
  MISC_FEATURE (9)..(9): Xaa is C or S
  MISC_FEATURE (10)..(10): Xaa is I or T
  MISC_FEATURE (11)..(11): Xaa is C or S
  MISC_FEATURE (12)..(12): Xaa is V or Y
  MISC_FEATURE (13)..(14): Xaa is K or R
XXXXXXXTXXXXXX

SEQ ID NO: 22 - protein, 39 aa, artificial
  DefMT3
GGYYCPFRQDKCHRHCRSFGRKAGYCGNFLKRTCICVKK

SEQ ID NO: 23 - protein, 38 aa, artificial
  DefMT6
GFGCPLNQGACHNHCRSIKRRGGYCSGIIKQTCTCYRK